(19) United States
(12) Patent Application Publication
(10) Pub. No.: US 2026/0092039 A1
(43) Pub. Date: Apr. 2, 2026

(54) DUAL INHIBITORS FOR THE TREATMENT OF ALZHEIMER'S DISEASE

(71) Applicants: UNIVERSITAT DE BARCELONA, Barcelona (ES); QPS CLINICAL SERVICES GMBH, Graz (AT)

(72) Inventors: Diego MUÑOZ-TORRERO LÓPEZ-IBARRA, Barcelona (ES); Santiago VÁZQUEZ CRUZ, Barcelona (ES); Noemí MARTÍNEZ CONDE, Barcelona (ES); Eugènia-Alexandra PUJOL BECH, Barcelona (ES); Tina LÖFFLER, Graz (AT); Daniela BRUNNER, Graz (AT); Manuela PROKESCH, Graz (AT); Roland RABL, Graz (AT); Birgit HUTTER-PAIER, Graz (AT)

(21) Appl. No.: 19/161,626

(22) PCT Filed: Mar. 4, 2024
(86) PCT No.: PCT/EP2024/055581

§ 371 Date: Sep. 2, 2025

(30) Foreign Application Priority Data
Mar. 3, 2023 (EP) 23382194.1

(51) Int. Cl. C07D 233/61; A61K 31/417; A61K 31/4178; C07D 493/18

(52) U.S. Cl. CPC C07D 233/61; A61K 31/417; A61K 31/4178; C07D 493/18

(57) ABSTRACT

Compounds (I) are provided, where R¹ and R² are H or (C1-C3)-alkyl; X is a linear methylene chain of formula —[CH₂]ₙ— with n=0, 1 or 2, or a biradical from a branched saturated (C2-C4)-alkylene chain; and A is either a C-radical from a non-aromatic polycyclic 6- to 15-membered carbocyclic ring system, or a C-radical from a polycyclic 6- to 15-membered heterocyclic ring system having one or two O, S or N; wherein the C-radicals are unsubstituted or substituted. Compounds (I) are simultaneously inhibitors of soluble epoxide hydrolase and inhibitors of glutaminyl cyclase. Besides, they reduce the levels of pro-inflammatory cytokines in LPS stimulated BV2 cells, display low cytotoxicity, and have good BBB permeability. Thus, they are useful as multitarget compounds for the prevention or treatment of Alzheimer's disease.

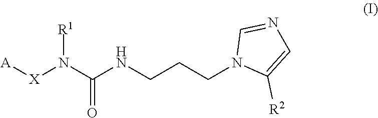

### TECHNICAL FIELD

The present invention relates to human therapy, particularly to the field of small-molecule compounds for multitarget treatment of Alzheimer's disease (AD).

### BACKGROUND ART

AD is a progressive neurodegenerative disorder that inevitably leads to the death of the patient after years of decline of cognitive and functional abilities, causing immense suffering to patients and caregivers. AD is the most common cause of dementia and the most prevalent neurodegenerative disorder. The devastating effects of AD can be ascribed, at least in part, to the lack of drugs that can prevent or arrest the progression of the disease. The few globally approved drugs just provide temporary symptomatic benefits. Approved drugs and most drug candidates in the drug development pipeline were designed to hit a single selected biological target, and most of the latter are failing in clinical trials.

These clinical failures may arise from a misconception of AD, which might be rather a complex pathological network, with several interconnected targets, that would remain resistant to change by modulation of just one single target. In other words, AD is not a matter of just one single biological target but, conversely, seems to result from a complex network where more than one biological target are playing a key role. In this scenario, new candidates with innovative mechanisms of action that can improve cognition and halt or delay AD progression are desperately needed. Multitarget drugs, i.e. single molecules that hit several targets of the complex AD network, have emerged as a realistic option to halt disease progression.

The AD drug discovery arena is one of the fields where the development of multitarget therapies has been most vigorously pursued in the past decade. The combination of two or more distinct pharmacophoric moieties in a single hybrid molecule represents the most usual way to build multitarget anti-Alzheimer drug candidates. Using this approach, a plethora of structural classes has been rationally designed, synthesized, and tested in vitro against the planned biological targets and, often, screened against additional proteins or pathological events of interest, but very frequently they are rather large molecules, with high molecular weight and lipophilicity, which results in poor physicochemical and pharmacokinetic properties. Also, in many cases these compounds were designed to hit biological targets involved in different disease stages (non contemporaneous). Indeed, no rationally designed multitarget compound has been approved so far for treatment of AD.

Several types of dual inhibitors have been designed that target the soluble epoxide hydrolase (sEH, EC 3.3.2.10) to take advantage of the beneficial effects of sEH inhibition on inflammation, one of the key early mechanisms of AD (for reviews, see e.g.: K. Hiesinger et al., “Development of multitarget agents possessing soluble epoxide hydrolase inhibitory activity”, Prostaglandins and Other Lipid Mediators, 2019, vol. 140, pp. 31-39; and M. R. Iyer, “Soluble epoxide hydrolase inhibitors: an overview and patent review from the last decade”, Expert Opinion on Therapeutic Patents, 2022, vol. 32, pp. 629-647).

Some of the present inventors discovered in 2018 that sEH inhibition was beneficial for the treatment of AD, and in 2019 they filed a patent application disclosing the use of dual sEH and acetylcholinesterase (AChE) inhibitors for the treatment of AD (cf. WO 2020/193448 A1). Up to now, however, none of these dual sEH/AChE inhibitors is being developed as a drug for AD treatment.

Single inhibitors of glutaminyl cyclase (QC, also known as glutaminyl-peptide cyclotransferase, QPCT, EC 2.3.2.5.) are known in the art (cf. e.g.: C. Xu et al., “Glutaminyl Cyclase, Diseases, and Development of Glutaminyl Cyclase Inhibitors”, Journal of Medicinal Chemistry 2021, vol. 64. pp. 6549-6565); and some of them have been proposed for the treatment of several diseases, including AD (cf. e.g.: J. Coimbra et al., “An overview of glutaminyl cyclase inhibitors for Alzheimer's disease”, Future Medicinal Chemistry 2019, vol. 11, pp. 3179-3194; and D. K. Vijayan et al., “Human glutaminyl cyclase: Structure, function, inhibitors and involvement in Alzheimer's disease”, Pharmacological Research 2019, vol. 147, 104342). All these QC inhibitors will likely have the limitations of previously developed single target anti-AD drug candidates.

Thus, the discovery of new candidates with innovative mechanisms of action and potential to halt or delay AD progression is both highly necessary and timely.

### SUMMARY OF INVENTION

Inventors have found that dual inhibition of sEH and glutaminyl cyclase (QC) has a synergistic action on the contemporaneous neuroinflammation and amyloid pathology, two crucial early mechanisms of AD. Thus, inventors disclose here what they believe is the first family of small-molecule compounds with dual activity on both sEH and QC, and drug-like properties, which hold great potential to halt or delay AD progress.

An aspect of the present disclosure relates to the provision of compounds of formula I, their stereoisomers and mixtures of stereoisomers, and the pharmaceutically acceptable salts of any of them,

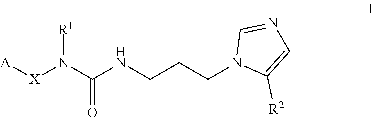

wherein:
R¹ and R² are each one a radical independently selected from the group consisting of H and (C1-C3)-alkyl;
X is a biradical selected from the group consisting of a biradical of a linear methylene chain of formula —[CH₂]ₙ— with n=0, 1 or 2, and a biradical from a branched saturated (C2-C4)-alkylene chain; and
A is either a C-radical from a non-aromatic polycyclic 6- to 15-membered carbocyclic ring system, or a C-radical from a non-aromatic polycyclic 6- to 15-membered heterocyclic ring system having one or two heteroatoms in the ring, the heteroatoms being independently selected from O, S and N; wherein the C-radicals are unsubstituted or the C-radicals have one or more of their H atoms substituted by one or more substituents that are attached to any of the possible substitution positions, the substituents being independently selected from the group consisting of (C1-C4)-alkyl, O[(C1-C4)-alkyl], OH, F, Cl, Br, and I;
on the proviso that radical A does not have any one of the three accompanying unsubstituted formulas, nor any of the possible substituted formulas derived thereof; and

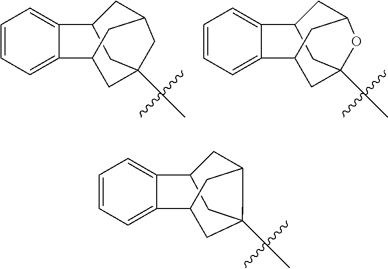 [SMILES: *C12CC3CC(C1)c1ccccc1C(C3)C2 ; *C12CC3CC(CC(C1)c1ccccc13)O2 ; *C12CC3CC1CC(C2)c1ccccc13]

on the proviso that compound I does not have the accompanying formula Ib.

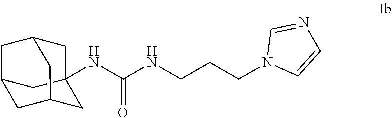 [SMILES: O=C(NCCCn1ccnc1)NC12CC3CC(CC(C3)C1)C2]

Compound Ib is here disclaimed because it has been already mentioned in the art, although in an unrelated context. In particular, compound Ib corresponds to the “compound 7” mentioned in the paper by J. T. Chenge et al., “Structural Characterization and Ligand/Inhibitor Identification Provide Functional Insights into the Mycobacterium tuberculosis Cytochrome P450 CYP126A1”, Journal of Biological Chemistry 2017, vol. 292, pp. 1310-1329. Its formula appears on p. 1315 of the paper, but its preparation is neither described, nor referred to. Concerning access to “compound 7”, the paper merely states that: “Novel compounds (compounds 1-9) were from the compound library of the Screening Facility at the Department of Medicinal Chemistry, Leibniz Institute of Molecular Pharmacology (Berlin, Germany) or sourced from ChemDiv or Vitas M.” (cf. section Materials at the end of p. 1326). “Compound 7” is compared, via high throughput screening (HTS) methods, with—among others—several antifungal drugs (ketoconazole, clotrimazole, econazole and miconazole), concluding that—similarly to the antifungals, but via a different mechanism—“compound 7” coordinates the cytochrome P450 CYP126A1. Nothing is known in the art about a specific therapeutical activity of “compound 7” (here named compound Ib). In the present disclosure, however, the preparation of compound Ib and a biological activity thereof are fully described (cf. Example 2 and Table 1).

In particular embodiments of compounds of formula I, radical A has a formula which is one of the five unsubstituted following formulas. In other particular embodiments, A has a formula which is a modification of the five following formulas in which one or more of the H atoms are substituted by one or more substituents, attached to any of the possible substitution positions, the substituents being (C1-C4)-alkyl, O[(C1-C4)-alkyl], OH, F, Cl, Br, or I. In other particular embodiments, the substituents are: CH₃, CH₂CH₃, OH, F, or Cl.

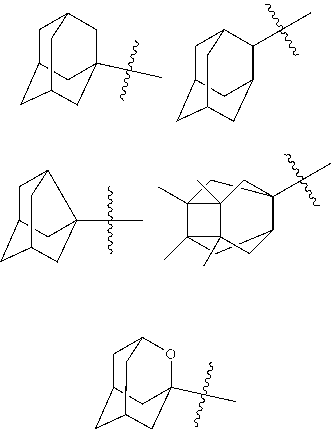 [SMILES: *C12CC3CC(CC(C3)C1)C2 ; *C1C2CC3CC(C2)CC1C3 ; *C12CC3CC(CC1C3)C2 ; *C12CC3(C)C4(C)CC1CC4(C)C3(C)C2 ; *C12CC3CC(CC(C3)O1)C2]

In particular embodiments of compounds of formula I, biradical X is either a linear methylene chain of formula —[CH₂]ₙ— with n=0, 1 or 2, or X is —CH(CH₃)—. In other particular embodiments, X is a linear methylene chain of formula —[CH₂]ₙ— with n=0, 1 or 2. And in other particular embodiments, n=0, biradical X being a single bond.

In particular embodiments of compounds of formula I, radical R¹ is H or CH₃. In other particular embodiments, R¹ is H.

In particular embodiments of compounds of formula I, radical R² is H or CH₃. In other particular embodiments, R² is CH₃.

In particular embodiments, the compounds of formula I have one of the formulas Ia, Ic, Id, Ie, If, Ig, Ih, Ii, Ij, Ik, Im, Io, Ip, Iq, Ir, Is, It, Iu, Iv, Iw, Ix, Iy, Iz, Iaa, Iab or Iac all of them shown in the present disclosure together with their respective preparation examples (cf. Examples 1 and 3-27).

Another aspect of the present disclosure relates to the provision of pharmaceutical compositions which comprise an effective amount of any of the above-defined compounds of formula I, together with adequate amounts of pharmaceutically acceptable excipients or carriers.

Another aspect of the present disclosure relates to any of the above-defined compounds of formula I for use as active pharmaceutical ingredients.

Another aspect of the present disclosure relates to any of the above-defined compounds of formula I, including the compound of formula Ib, or any stereoisomer, or any stereoisomer mixture, or any pharmaceutically acceptable salt thereof, for use in the prevention or treatment of Alzheimer's disease in an animal, including a human. This aspect also relates to the use of any of the above-defined compounds of formula I including compound Ib, in the preparation of a medicine for the prevention or treatment of AD in an animal, including a human. It may be said that this aspect relates to a method of prevention or treatment of AD in an animal, including a human, that comprises the administration, to the animal or the human, of a therapeutically effective amount of any of the above-defined compounds of formula I including the compound of formula Ib, together with adequate amounts of pharmaceutically acceptable excipients or carriers.

As illustrated in accompanying preparation examples, compounds of formula I can be prepared by processes which are known in general terms, from starting materials which either are known, or are available via analogy processes from known materials. Thus, compounds I where R¹=H can be prepared by a process which comprises reacting corresponding isocyanates of formula II with corresponding amines of formula III or their salts. If desired, compounds of formula I thereby obtained may be converted into their pharmaceutically acceptable salts by reacting with corresponding pharmaceutically acceptable acids.

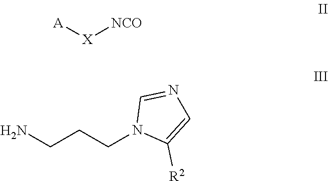

Alternatively, compounds of formula I where R¹=H can be prepared by a process which comprises reacting corresponding isocyanates of formula V with corresponding amines of formula IV or their salts.

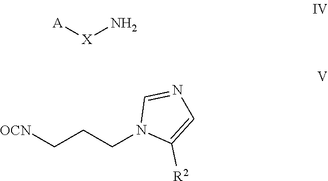

Intermediate isocyanates of formulas II and V can be prepared by subjecting corresponding primary amines of formulas IV and III, respectively, to a phosgenation reaction, using phosgene, diphosgene or triphosgene.

Alternatively, compounds of formula I where R¹=H can be prepared by a process which comprises reacting corresponding amines of formula III with 1,1′-carbonyldiimidazole, followed by reaction with corresponding amines of formula IV, or by a process which comprises reacting corresponding amines of formula IV with 1,1′-carbonyldiimidazole, followed by reaction with corresponding amines of formula III.

Compounds of formula I where R¹=(C1-C3)-alkyl can be prepared by a process which comprises reacting corresponding amines of formula IV′ where R¹=(C1-C3)-alkyl, with corresponding azides of formula VI, triphenylphosphine and carbon dioxide.

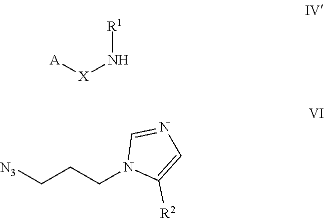

As illustrated by the results of the in vitro assays of accompanying examples, compounds I of the present invention or their pharmaceutically acceptable salts are simultaneously inhibitors of sEH (cf. Example 28) and inhibitors of QC (cf. Example 29). Besides, they reduce the levels of pro-inflammatory cytokines in LPS stimulated BV2 cells (cf. Example 30), display low cytotoxicity (cf. Example 31) and have good blood-brain barrier (BBB) permeability (cf. Example 32). Thus, compounds I or their salts are useful as multitarget agents for the prevention or treatment of AD.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### DESCRIPTION OF EMBODIMENTS

Melting points were determined in open capillary tubes with a MFB 595010M Gallenkamp melting point apparatus. High resolution mass spectra were carried out at the Centres Cientifics i Tecnològics of the University of Barcelona with a LC/MSD TOF Agilent Technologies spectrometer. IR spectra were run on a Perkin-Elmer Spectrum RX I spectrophotometer. Absorption values are expressed as wavenumbers (cm⁻¹). Column chromatography was performed on silica gel 60 AC.C (40-60 mesh, Carlo Erba, ref. 2000027). Automatic normal phase column chromatography was performed on a CombiFlash Rf 150 (Teledyne Isco) with pre-packed RediSep Rf silica gel cartridges. Thin-layer chromatography was performed with silica gel 60 F254 (Merck, ref. 1.05554), and spots were visualized with 1% aqueous solution of KMnO₄. Pure for synthesis solvents were used in the reactions, extractions and column chromatography.

### Example 1: Preparation of 1-(adamantan-1-yl)-3-[3-(5-methyl-1H-imidazol-1-yl)propyl]urea, of Formula Ia

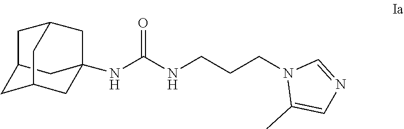 [SMILES: Cc1cncn1CCCNC(=O)NC12CC3CC(CC(C3)C1)C2]

1-Adamantylamine hydrochloride (282 mg, 1.50 mmol) was added to a stirred mixture of dichloromethane (6 mL) and saturated aqueous NaHCO₃ (8 mL). Triphosgene (223 mg, 0.75 mmol) was then slowly added and the reaction mixture was stirred at room temperature for 30 min. Thereafter, the two phases were separated and the organic layer was washed with brine, dried over anhydrous Na₂SO₄ and filtered. The filtrate was partially evaporated at reduced pressure below 30° C., to provide a concentrated dichloromethane solution of 1-adamantyl isocyanate, which was used in the following step without further purification.

To the solution of the isocyanate, a solution of 3-(5-methyl-1H-imidazol-1-yl)propan-1-amine (251 mg, 1.80 mmol) in dichloromethane (1 mL) was added. The reaction mixture was stirred at room temperature overnight and was evaporated at reduced pressure to give a beige gum (359 mg), which was purified by column chromatography (40-60 μm silica gel, dichloromethane-methanol mixtures), to afford the title compound (222 mg, 47% overall yield) as a yellow gummy solid.

Characterization of Ia: mp 80-81° C.; IR (NaCl) v: 3316, 2903, 2847, 1657, 1565, 1504, 1451, 1358, 1293, 1277, 1233, 1109, 1093, 928, 822, 753, 666, 630 cm⁻¹; HRMS (ESI) calcd for [C₁₃H₂₃N₄O+H]⁺ 317.2336, found 317.2331.

### Example 2: Preparation of 1-[3-(1H-imidazol-1-yl)propyl]-3-(adamantan-1-yl)urea, of Formula Ib (Prior Art)

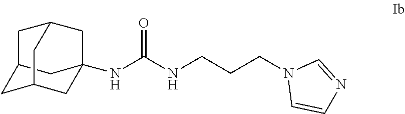 [SMILES: O=C(NCCCn1ccnc1)NC12CC3CC(CC(C3)C1)C2]

It was prepared in an analogous manner to the one described for the compound of Example 1. Starting from 1-adamantylamine hydrochloride (282 mg, 1.50 mmol) and triphosgene (223 mg, 0.75 mmol) and reacting the resulting dichloromethane concentrated solution of 1-adamantyl isocyanate with 3-(1H-imidazol-1-yl)propan-1-amine (225 mg, 1.80 mmol), a yellowish solid residue (500 mg) was obtained. Column chromatography purification of this residue (40-60 μm silica gel, dichloromethane-methanol mixtures) afforded the title compound (150 mg, 33% overall yield).

Characterization of Ib: mp 127-129° C.; IR (NaCl) v: 3354, 3113, 3021, 2907, 2848, 1624, 1563, 1504, 1452, 1356, 1293, 1280, 1241, 1229, 1104, 1090, 1074, 905, 807, 661, 621 cm⁻¹; HRMS (ESI) calcd for [C₁₇H₂₆N₄O+H]⁺ 303.2179, found 303.2177.

### Example 3: Preparation of 1-[3-(5-methyl-1H-imidazol-1-yl)propyl]-3-(3-methyladamantan-1-yl)urea, of Formula Ic

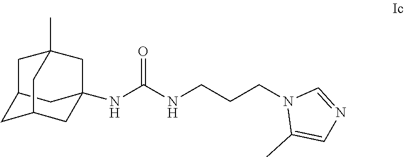 [SMILES: Cc1cncn1CCCNC(=O)NC12CC3CC(CC(C)(C3)C1)C2]

It was prepared in an analogous manner to the one described for the compound of Example 1. Starting from (3-methyl-1-adamantyl)amine hydrochloride (242 mg, 1.20 mmol) and triphosgene (178 mg, 0.60 mmol) and reacting the resulting dichloromethane concentrated solution of 3-methyl-1-adamantyl isocyanate with 3-(5-methyl-1H-imidazol-1-yl)propan-1-amine (200 mg, 1.44 mmol), a yellowish residue (346 mg) was obtained. Column chromatography purification of this residue (40-60 μm silica gel, dichloromethane-methanol mixtures), followed by washing with pentane (3×2 mL) and drying at 20 Torr for 48 h, afforded the title compound (188 mg, 48% overall yield) as a white solid.

Characterization of Ic: mp 71-73° C.; IR (ATR) v: 3446, 3354, 3183, 3113, 2912, 2838, 1659, 1563, 1497, 1445, 1432, 1355, 1306, 1263, 1238, 1206, 1155, 1105, 1055, 929, 851, 833, 802, 700, 666 cm⁻¹; HRMS (ESI) calcd for [C₁₉H₃₀N₄O+H]⁺ 331.2492, found 331.2493.

### Example 4: Preparation of 1-(3-ethyladamantan-1-yl)-3-[3-(5-methyl-1H-imidazol-1-yl)propyl]urea, of Formula Id

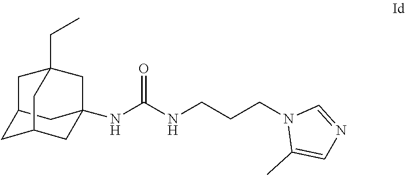 [SMILES: CCC12CC3CC(C1)CC(NC(=O)NCCCn1cncc1C)(C3)C2]

It was prepared in an analogous manner to the one described for the compound of Example 1. Starting from 3-ethyladamantylamine hydrochloride (324 mg, 1.50 mmol) and triphosgene (223 mg, 0.75 mmol) and reacting the resulting dichloromethane concentrated solution of 3-ethyl-1-adamantyl isocyanate with 3-(5-methyl-1H-imidazol-1-yl)propan-1-amine (251 mg, 1.80 mmol), a yellowish gum (494 mg) was obtained. Column chromatography purification of this residue (40-60 μm silica gel, dichloromethane-methanol mixtures), afforded the title compound (307 mg, 59% overall yield) as a yellowish gum.

Characterization of Id: IR (NaCl) v: 3345, 3110, 2960, 2908, 2848, 1645, 1562, 1505, 1451, 1360, 1297, 1246, 1235, 1110, 928, 808 cm⁻¹; HRMS (ESI) calcd for [C₂₀H₃₂N₄O+H]⁺ 345.2649, found 345.2651.

### Example 5: Preparation of 1-(3,5-dimethyladamantan-1-yl)-3-[3-(5-methyl-1H-imidazol-1-yl)propyl]urea, of Formula Ie

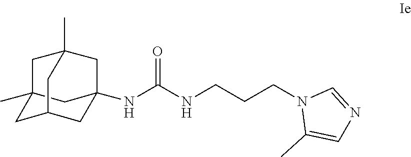 [SMILES: Cc1cncn1CCCNC(=O)NC12CC3CC(C)(CC(C)(C3)C1)C2]

It was prepared in an analogous manner to the one described for the compound of Example 1. Starting from (3,5-dimethyl-1-adamantyl)amine hydrochloride (117 mg, 0.54 mmol) and triphosgene (80 mg, 0.27 mmol) and reacting the resulting dichloromethane concentrated solution of 3,5-dimethyl-1-adamantyl isocyanate with 3-(5-methyl-1H-imidazol-1-yl)propan-1-amine (91 mg, 0.65 mmol), a yellowish residue (158 mg) was obtained. After two consecutive column chromatography purifications (40-60 μm silica gel, dichloromethane-methanol mixtures), followed by washing with pentane (3×2 mL) and drying at 20 Torr for 48 h, the title compound (65 mg, 35% overall yield) was obtained as a white solid.

Characterization of Ie: mp 112-114° C.; IR (ATR) v: 3332, 3197, 3099, 3042, 2944, 2900, 2839, 1683, 1556, 1505, 1453, 1357, 1263, 1233, 1206, 1107, 931, 820, 693, 666, 626 cm⁻¹; HRMS (ESI) calcd for [C₂₀H₃₂N₄O+H]⁺ 345.2649, found 345.2647.

### Example 6: Preparation of 1-(3-chloroadamantan-1-yl)-3-[3-(5-methyl-1H-imidazol-1-yl)propyl]urea, of Formula If

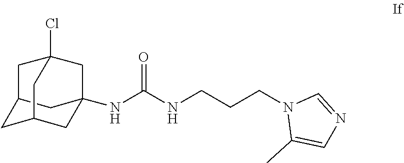 [SMILES: Cc1cncn1CCCNC(=O)NC12CC3CC(CC(Cl)(C3)C1)C2]

It was prepared in an analogous manner to the one described for the compound of Example 1. Starting from 3-chloroadamantan-1-amine hydrochloride (166 mg, 0.75 mmol) and triphosgene (133 mg, 0.45 mmol) and reacting the resulting dichloromethane concentrated solution of 3-chloro-1-adamantyl isocyanate with 3-(5-methyl-1H-imidazol-1-yl)propan-1-amine (149 mg, 1.07 mmol), a yellowish residue (231 mg) was obtained. The residue was purified by column chromatography (40-60 μm silica gel, dichloromethane-methanol mixtures), followed by washing with pentane (3×2 mL) and drying at 20 Torr for 48 h, to afford the title compound (55 mg, 21% overall yield) as a brown solid.

Characterization of If: mp 110-112° C.; IR (ATR) v: 3302, 3115, 3042, 2913, 2855, 1667, 1645, 1638, 1557, 1525, 1452, 1390, 1369, 1351, 1333, 1291, 1255, 1227, 1202, 1184, 1135, 1109, 1096, 1043, 977, 962, 939, 833, 802, 756, 725, 695, 657, 634 cm⁻¹; HRMS (ESI) calcd for [C₁₈H₂₇ClN₄O+H]⁺ 351.1946, found 351.1949.

### Example 7: Preparation of 1-(3-fluoroadamantan-1-yl)-3-[3-(5-methyl-1H-imidazol-1-yl)propyl]urea, of Formula Ig

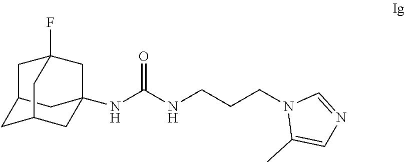 [SMILES: Cc1cncn1CCCNC(=O)NC12CC3CC(CC(F)(C3)C1)C2]

It was prepared in an analogous manner to the one described for the compound of Example 1. Starting from 3-fluoroadamantan-1-amine (240 mg, 1.42 mmol) and triphosgene (211 mg, 0.71 mmol) and reacting the resulting dichloromethane concentrated solution of 3-fluoro-1-adamantyl isocyanate with 3-(5-methyl-1H-imidazol-1-yl)propan-1-amine (237 mg, 1.70 mmol), a yellowish residue (517 mg) was obtained. The residue was purified by column chromatography (40-60 μm silica gel, dichloromethane-methanol mixtures), followed by washing with pentane (3×2 mL) and drying at 20 Torr for 48 h, to afford the title compound (42 mg, 9% overall yield) as a dark yellow solid.

Characterization of Ig: mp 115-117° C.; IR (ATR) v: 3456, 3357, 3316, 3123, 2914, 2860, 1659, 1562, 1557, 1522, 1454, 1442, 1425, 1363, 1352, 1330, 1290, 1260, 1237, 1202, 1108, 1018, 1012, 947, 929, 903, 841, 825, 797, 718, 695, 667, 656, 607 cm⁻¹; HRMS (ESI) calcd for [C₁₈H₂₇FN₄O+H]⁺ 335.2242, found 335.2245.

### Example 8: Preparation of 1-(3-hydroxyadamantan-1-yl)-3-[3-(5-methyl-1H-imidazol-1-yl)propyl]urea, of Formula Ih

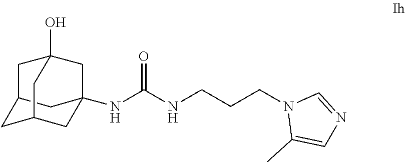 [SMILES: Cc1cncn1CCCNC(=O)NC12CC3CC(CC(O)(C3)C1)C2]

It was prepared in an analogous manner to the one described for the compound of Example 1. Starting from 3-amino-1-adamantanol (206 mg, 1.23 mmol) and triphosgene (183 mg, 0.62 mmol) and reacting the resulting dichloromethane concentrated solution of 3-hydroxy-1-adamantyl isocyanate with 3-(5-methyl-1H-imidazol-1-yl)propan-1-amine (206 mg, 1.48 mmol), a yellowish residue (307 mg) was obtained. The residue was subjected to two consecutive column chromatography purifications (40-60 μm silica gel, dichloromethane-methanol mixtures), followed by washing with pentane (3×1 mL) and drying at 20 Torr for 48 h, to afford the title compound (53 mg, 13% overall yield) as a beige solid.

Characterization of Ih: mp 52-54° C.; IR (ATR) v: 3311, 2968, 2909, 2852, 1646, 1556, 1503, 1449, 1390, 1353, 1332, 1290, 1231, 1122, 1107, 1048, 928, 910, 804, 715, 663, 627 cm⁻¹; HRMS (ESI) calcd for [C₁₃H₂₃N₄O₂+H]⁺ 333.2285, found 333.2281.

### Example 9: Preparation of 1-(adamantan-2-yl)-3-[3-(5-methyl-1H-imidazol-1-yl)propyl]urea, of Formula Ii

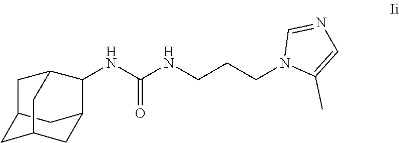 [SMILES: Cc1cncn1CCCNC(=O)NC1C2CC3CC(C2)CC1C3]

It was prepared in an analogous manner to the one described for the compound of Example 1. Starting from 2-adamantanamine hydrochloride (282 mg, 1.50 mmol) and triphosgene (223 mg, 0.75 mmol) and reacting the resulting dichloromethane concentrated solution of 2-adamantyl isocyanate with 3-(5-methyl-1H-imidazol-1-yl)propan-1-amine (251 mg, 1.80 mmol), an orange gummy residue (483 mg) was obtained. The residue was purified by column chromatography (40-60 μm silica gel, dichloromethane-methanol mixtures), to afford the title compound (285 mg, 60% overall yield) as a beige solid.

Characterization of Ii: mp 76-77° C.; IR (ATR) v: 3320, 2904, 2850, 1674, 1563, 1557, 1511, 1465, 1449, 1366, 1274, 1246, 1233, 1209, 1106, 1063, 931, 814, 803 cm⁻¹; HRMS (ESI) calcd for [C₁₈H₂₈N₄O+H]⁺ 317.2336, found 317.2335.

### Example 10: Preparation of 1-[3-(1H-imidazol-1-yl)propyl]-3-(adamantan-2-yl)urea, of Formula Ij

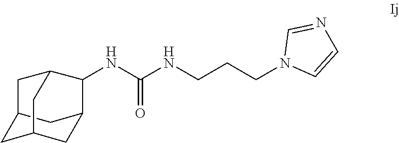 [SMILES: O=C(NCCCn1ccnc1)NC1C2CC3CC(C2)CC1C3]

It was prepared in an analogous manner to the one described for the compound of Example 1. Starting from 2-adamantanamine hydrochloride (1.04 g, 5.52 mmol) and triphosgene (820 mg, 2.76 mmol) and reacting the resulting dichloromethane concentrated solution of 2-adamantyl isocyanate with 3-(1H-imidazol-1-yl)propan-1-amine (0.75 mL, 6.39 mmol), an orange gummy residue (3.11 g) was obtained. The residue was purified by column chromatography (40-60 μm silica gel, dichloromethane-methanol mixtures), followed by washing with pentane (3×3 mL) and drying at 20 Torr for 48 h to afford the title compound (1.08 g, 67% overall yield) as a white solid.

Characterization of Ij: mp 144-146° C.; IR (ATR) v: 3395, 3264, 3101, 2903, 2885, 2850, 1662, 1554, 1517, 1500, 1470, 1450, 1440, 1390, 1361, 1346, 1321, 1295, 1278, 1256, 1235, 1106, 1096, 1078, 1064, 1042, 1029, 1000, 971, 963, 915, 872, 839, 814, 741, 670, 641, 627 cm⁻¹; HRMS (ESI) calcd for [C₁₇H₂₆N₄O+H]⁺ 303.2179, found 303.2179.

### Example 11: Preparation of 1-[(adamantan-1-yl)methyl]-3-[3-(5-methyl-1H-imidazol-1-yl)propyl]urea, of Formula Ik

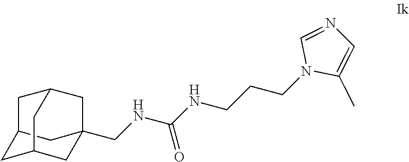 [SMILES: Cc1cncn1CCCNC(=O)NCC12CC3CC(CC(C3)C1)C2]

It was prepared in an analogous manner to the one described for the compound of Example 1. Starting from (adamantan-1-yl)methanamine (190 mg, 1.15 mmol) and triphosgene (171 mg, 0.57 mmol) and reacting the resulting dichloromethane concentrated solution of (adamantan-1-yl)methyl isocyanate with 3-(5-methyl-1H-imidazol-1-yl)propan-1-amine (192 mg, 1.38 mmol), a yellowish residue (387 mg) was obtained. The residue was purified by column chromatography (40-60 μm silica gel, dichloromethane-methanol mixtures), followed by washing with pentane (3×3 mL) and drying at 20 Torr for 48 h, to afford the title compound (148 mg, 39% overall yield) as a brown sticky oil.

Characterization of Ik: IR (ATR) v: 3323, 3113, 3043, 2898, 2845, 1639, 1563, 1503, 1448, 1394, 1364, 1346, 1310, 1281, 1264, 1233, 1187, 1152, 1107, 1065, 983, 965, 925, 806, 732, 664 cm⁻¹; HRMS (ESI) calcd for [C₁₉H₃₀N₄O+H]⁺ 331.2492, found 331.2496.

### Example 12: Preparation of 1-[3-(1H-imidazol-1-yl)propyl]-3-[(adamantan-1-yl)methyl]urea, of Formula Im

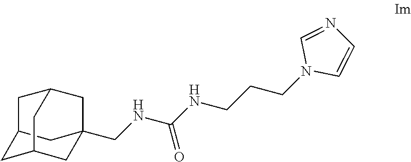 [SMILES: O=C(NCCCn1ccnc1)NCC12CC3CC(CC(C3)C1)C2]

It was prepared in an analogous manner to the one described for the compound of Example 1. Starting from (adamantan-1-yl)methanamine (178 mg, 1.08 mmol) and triphosgene (160 mg, 0.54 mmol) and reacting the resulting dichloromethane concentrated solution of (adamantan-1-yl)methyl isocyanate with 3-(1H-imidazol-1-yl)propan-1-amine (0.15 mL, 1.29 mmol), a yellowish solid residue (526 mg) was obtained. The residue was purified by column chromatography (40-60 μm silica gel, dichloromethane-methanol mixtures), followed by washing with pentane (3×3 mL) and drying at 20 Torr for 48 h, to afford the title compound (110 mg, 32% overall yield) as a light yellow sticky oil.

Characterization of Im: IR (ATR) v: 3322, 3110, 2898, 2845, 1634, 1563, 1507, 1449, 1399, 1363, 1346, 1310, 1285, 1230, 1188, 1107, 1079, 1032, 988, 975, 915, 812, 728, 662, 622 cm⁻¹; HRMS (ESI) calcd for [C₁₈H₂₈N₄O+H]⁺ 317.2336, found 317.2337.

### Example 13: Preparation of 1-[2-(adamantan-1-yl)ethyl]-3-[3-(5-methyl-1H-imidazol-1-yl)propyl]urea, of Formula Io

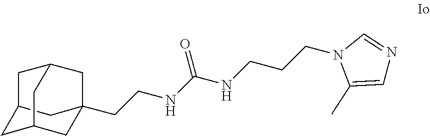 [SMILES: Cc1cncn1CCCNC(=O)NCCC12CC3CC(CC(C3)C1)C2]

It was prepared in an analogous manner to the one described for the compound of Example 1. Starting from 2-(adamantan-1-yl)ethan-1-amine (250 mg, 1.39 mmol) and triphosgene (207 mg, 0.70 mmol) and reacting the resulting dichloromethane concentrated solution of 2-(adamantan-1-yl)ethyl isocyanate with 3-(5-methyl-1H-imidazol-1-yl)propan-1-amine (233 mg, 1.67 mmol), a yellowish residue (443 mg) was obtained. The residue was subjected to two consecutive column chromatography purifications (40-60 μm silica gel, dichloromethane-methanol mixtures), followed by washing with pentane (3×2 mL) and drying at 20 Torr for 48 h, to afford the title compound (95 mg, 20% overall yield) as a colorless oil.

Characterization of Io: IR (ATR) v: 3308, 3114, 2898, 2845, 1639, 1563, 1503, 1448, 1387, 1364, 1355, 1312, 1265, 1237, 1215, 1194, 1167, 1108, 1071, 989, 967, 956, 925, 809, 733, 693, 664 cm⁻¹; HRMS (ESI) calcd for [C₂₀H₃₂N₄O+H]⁺ 345.2649, found 345.2649.

### Example 14: Preparation of 1-[2-(adamantan-2-yl)ethyl]-3-[3-(5-methyl-1H-imidazol-1-yl)propyl]urea, of Formula Ip

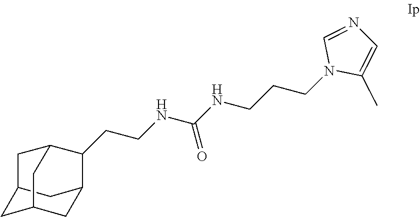 [SMILES: Cc1cncn1CCCNC(=O)NCCC1C2CC3CC(C2)CC1C3]

It was prepared in an analogous manner to the one described for the compound of Example 1. Starting from 2-(adamantan-2-yl)ethan-1-amine hydrochloride (192 mg, 0.89 mmol and triphosgene (132 mg, 0.44 mmol) and reacting the resulting dichloromethane concentrated solution of 2-(adamantan-2-yl)ethyl isocyanate with 3-(5-methyl-1H-imidazol-1-yl)propan-1-amine (149 mg, 1.07 mmol), a brown sticky residue (323 mg) was obtained. The residue was purified by column chromatography (40-60 μm silica gel, dichloromethane-methanol mixtures), followed by washing with pentane (3×3 mL) and drying at 20 Torr for 48 h, to afford the title compound (135 mg, 44% overall yield) as a yellowish sticky semi-solid.

Characterization of Ip: IR (ATR) v: 3301, 3102, 2900, 2848, 2676, 1638, 1557, 1499, 1447, 1388, 1365, 1353, 1245, 1232, 1207, 1176, 1107, 1072, 921, 806, 771, 731, 664, 625 cm⁻¹; HRMS (ESI) calcd for [C₂₀H₃₂N₄O+H]⁺ 345.2649, found 345.2652.

### Example 15: Preparation of 1-(2-oxaadamantan-1-yl)-3-[3-(5-methyl-1H-imidazol-1-yl)propyl]urea, of Formula Iq

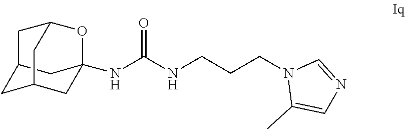 [SMILES: Cc1cncn1CCCNC(=O)NC12CC3CC(CC(C3)O1)C2]

It was prepared in an analogous manner to the one described for the compound of Example 1. Starting from 2-oxaadamantan-1-amine hydrochloride (284 mg, 1.50 mmol) and triphosgene (223 mg, 0.75 mmol) and reacting the resulting dichloromethane concentrated solution of 2-oxaadamantan-1-yl isocyanate with 3-(5-methyl-1H-imidazol-1-yl)propan-1-amine (251 mg, 1.80 mmol), a yellowish gummy residue (359 mg) was obtained. The residue was purified by column chromatography (40-60 μm silica gel, dichloromethane-methanol mixtures), to afford the title compound (167 mg, 35% overall yield) as a beige gum.

Characterization of Iq: IR (NaCl) v: 3329, 2926, 2852, 1659, 1561, 1504, 1447, 1377, 1324, 1275, 1262, 1195, 1118, 1093, 994, 965, 765, 750 cm⁻¹; HRMS (ESI) calcd for [C₁₇H₂₆N₄O₂+H]⁺ 319.2120, found 319.2132.

### Example 16: Preparation of 1-[3-(5-methyl-1H-imidazol-1-yl)propyl]-3-(3-methyl-2-oxaadamantan-1-yl)urea, of Formula Ir

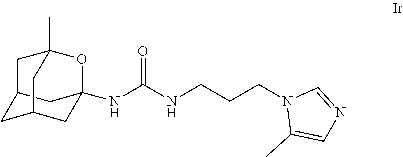 [SMILES: Cc1cncn1CCCNC(=O)NC12CC3CC(CC(C)(C3)O1)C2]

It was prepared in an analogous manner to the one described for the compound of Example 1. Starting from 3-methyl-2-oxaadamantan-1-amine hydrochloride (203 mg, 1.00 mmol) and triphosgene (148 mg, 0.50 mmol) and reacting the resulting dichloromethane concentrated solution of 3-methyl-2-oxaadamantan-1-yl isocyanate with 3-(5-methyl-1H-imidazol-1-yl)propan-1-amine (139 mg, 1.00 mmol), an orange gummy residue (282 mg) was obtained. The residue was purified by column chromatography (40-60 μm silica gel, dichloromethane-methanol mixtures), to afford the title compound (70 mg, 21% overall yield) as a beige gum.

Characterization of Ir: IR (NaCl) v: 3311, 3111, 3059, 2967, 2924, 2851, 1680, 1557, 1449, 1376, 1340, 1324, 1302, 1259, 1218, 1192, 1143, 1072, 1035, 994, 949, 920, 895 cm⁻¹; HRMS (ESI) calcd for [C₁₈H₂₈N₄O₂+H]⁺ 333.2285, found 333.2295.

### Example 17: Preparation of 1-(3-ethyl-2-oxaadamantan-1-yl)-3-[3-(5-methyl-1H-imidazol-1-yl)propyl]urea, of Formula Is

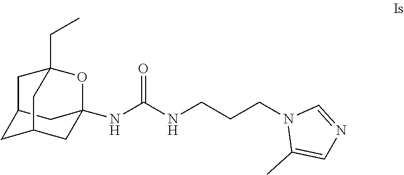 [SMILES: CCC12CC3CC(C1)CC(NC(=O)NCCCn1cncc1C)(C3)O2]

It was prepared in an analogous manner to the one described for the compound of Example 1. Starting from 3-ethyl-2-oxaadamantan-1-amine hydrochloride (79 mg, 0.36 mmol) and triphosgene (54 mg, 0.18 mmol) and reacting the resulting dichloromethane concentrated solution of 3-ethyl-2-oxaadamantan-1-yl isocyanate with 3-(5-methyl-1H-imidazol-1-yl)propan-1-amine (50 mg, 0.36 mmol), an orange gummy residue (124 mg) was obtained. The residue was purified by column chromatography (40-60 μm silica gel, dichloromethane-methanol mixtures), to afford the title compound (70 mg, 56% overall yield) as a yellowish gum.

Characterization of Is: IR (NaCl) v: 3358, 3100, 2926, 2853, 1673, 1551, 1501, 1447, 1375, 1298, 1259, 1208, 1109, 1074, 1005, 969, 940, 897, 805 cm⁻¹; HRMS (ESI) calcd for [C₁₉H₃₀N₄O₂+H]⁺ 347.2442, found 347.2447.

### Example 18: Preparation of 1-(noradamantan-3-yl)-3-[3-(5-methyl-1H-imidazol-1-yl)propyl]urea, of Formula It

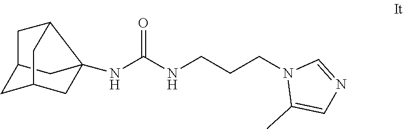 [SMILES: Cc1cncn1CCCNC(=O)NC12CC3CC(CC1C3)C2]

It was prepared in an analogous manner to the one described for the compound of Example 1. Starting from 3-noradamantanamine hydrochloride (200 mg, 1.15 mmol) and triphosgene (171 mg, 0.58 mmol) and reacting the resulting dichloromethane concentrated solution of 3-noradamantyl isocyanate with 3-(5-methyl-1H-imidazol-1-yl)propan-1-amine (192 mg, 1.38 mmol), a yellowish residue (303 mg) was obtained. The residue was purified by column chromatography (40-60 μm silica gel, dichloromethane-methanol mixtures), followed by washing with pentane (3×3 mL) and drying at 20 Torr for 48 h, to afford the title compound (156 mg, 45% overall yield) as a white solid.

Characterization of It: mp 126-128° C.; IR (ATR) v: 3368, 3314, 2955, 2931, 2906, 2865, 2839, 1629, 1562, 1529, 1500, 1445, 1365, 1348, 1329, 1308, 1295, 1282, 1262, 1245, 1226, 1205, 1191, 1143, 1136, 1118, 1106, 1092, 1056, 1000, 918, 860, 844, 826, 806, 795, 778, 724, 697, 688, 662, 647 cm⁻¹; HRMS (ESI) calcd for [C₁₇H₂₆N₄O+H]⁺ 303.2179, found 303.2187.

### Example 19: Preparation of 1-[(noradamantan-3-yl)methyl]-3-[3-(5-methyl-1H-imidazol-1-yl)propyl]urea, of Formula Iu

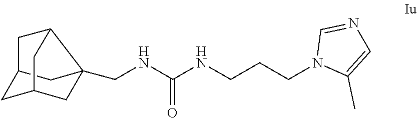 [SMILES: Cc1cncn1CCCNC(=O)NCC12CC3CC(CC1C3)C2]

It was prepared in an analogous manner to the one described for the compound of Example 1. Starting from (noradamantan-3-yl)methanamine hydrochloride (220 mg, 1.17 mmol) and triphosgene (174 mg, 0.59 mmol) and reacting the resulting dichloromethane concentrated solution of (noradamantan-3-yl)methyl isocyanate with 3-(5-methyl-1H-imidazol-1-yl)propan-1-amine (196 mg, 1.41 mmol), a yellow residue (318 mg) was obtained. Column chromatography purification of this residue (40-60 μm silica gel, dichloromethane-methanol mixtures), followed by washing with pentane (3×3 mL) and drying at 20 Torr for 48 h, afforded the title compound (206 mg, 56% overall yield) as a yellow sticky gum.

Characterization of Iu: IR (ATR) v: 3313, 3109, 2918, 2860, 1635, 1557, 1500, 1445, 1388, 1364, 1331, 1309, 1232, 1124, 1108, 1077, 995, 921, 863, 806, 773, 730, 664 cm⁻¹; HRMS (ESI) calcd for [C₁₈H₂₈N₄O+H]⁺ 317.2336, found 317.2339.

### Example 20: Preparation of 1-[3-(5-methyl-1H-imidazol-1-yl)propyl]-3-(3,4,8,9-tetramethyltetracyclo[4.4.0.0^{3,9}.0^{4,8}]decan-1-yl)urea, of Formula Iv

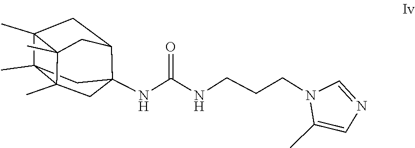 [SMILES: Cc1cncn1CCCNC(=O)NC12CC3(C)C4(C)CC1CC4(C)C3(C)C2]

It was prepared in an analogous manner to the one described for the compound of Example 1. Starting from 3,4,8,9-tetramethyltetracyclo[4.4.0.0^{3,9}.0^{4,8}]decan-1-amine hydrochloride (119 mg, 0.58 mmol) and triphosgene (86 mg, 0.29 mmol) and reacting the resulting dichloromethane concentrated solution of 3,4,8,9-tetramethyltetracyclo[4.4.0.0^{3,9}.0^{4,8}]decan-1-yl isocyanate with 3-(5-methyl-1H-imidazol-1-yl)propan-1-amine (97 mg, 0.70 mmol), a yellowish residue (167 mg) was obtained. The residue was purified by column chromatography (40-60 μm silica gel, dichloromethane-methanol mixtures), followed by washing with pentane (3×1 mL) and drying at 20 Torr for 48 h, to afford the title compound (87 mg, 41% overall yield) as a white solid.

Characterization of Iu: mp 130-133° C.; IR (ATR) v: 3279, 3121, 2972, 2944, 2908, 2864, 1675, 1556, 1505, 1478, 1454, 1439, 1384, 1368, 1335, 1315, 1290, 1262, 1243, 1234, 1208, 1108, 1062, 1033, 933, 847, 803, 793, 766, 728, 697, 666, 631 cm⁻¹; HRMS (ESI) calcd for [C₂₂H₃₄N₄O+H]⁺ 371.2805, found 371.2815.

### Example 21: Preparation of 1-[3-(5-methyl-1H-imidazol-1-yl)propyl]-3-[(3,4,8,9-tetramethyltetracyclo[4.4.0.0^{3,9}.0^{4,8}]decan-1-yl)methyl]urea, of Formula Iw

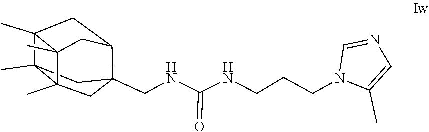 [SMILES: Cc1cncn1CCCNC(=O)NCC12CC3(C)C4(C)CC1CC4(C)C3(C)C2]

It was prepared in an analogous manner to the one described for the compound of Example 1. Starting from (3,4,8,9-tetramethyltetracyclo[4.4.0.0^{3,9}.0^{4,8}]decan-1-yl)methanamine hydrochloride (42 mg, 0.16 mmol) and triphosgene (24 mg, 0.08 mmol) and reacting the resulting dichloromethane concentrated solution of (3,4,8,9-tetramethyltetracyclo[4.4.0.0^{3,9}.0^{4,8}]decan-1-yl)methyl isocyanate with 3-(5-methyl-1H-imidazol-1-yl)propan-1-amine (27 mg, 0.20 mmol), a light orange residue (79 mg) was obtained. The residue was purified by column chromatography (40-60 μm silica gel, dichloromethane-methanol mixtures), followed by washing with pentane (3×0.5 mL) and drying at 20 Torr for 48 h, to afford the title compound (10 mg, 16% overall yield) as a yellowish semi-solid.

Characterization of Iw: IR (NaCl) v: 3302, 3111, 3045, 2944, 2863, 1723, 1647, 1567, 1503, 1453, 1385, 1371, 1340, 1262, 1109, 1076, 1038, 927, 807, 775, 732, 700, 665, 637 cm⁻¹; HRMS (ESI) calcd for [C₂₃H₃₆N₄O+H]⁺ 385.2962, found 385.2962.

### Example 22: Preparation of 1-[1-(adamantan-1-yl)ethyl]-3-[3-(5-methyl-1H-imidazol-1-yl)propyl]urea, of Formula Ix

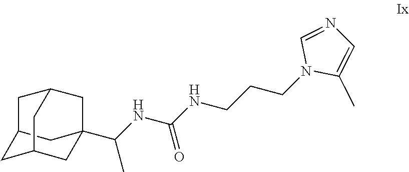 [SMILES: Cc1cncn1CCCNC(=O)NC(C)C12CC3CC(CC(C3)C1)C2]

It was prepared in an analogous manner to the one described for the compound of Example 1. Starting from 1-(adamantan-1-yl)ethan-1-amine hydrochloride (247 mg, 1.14 mmol) and triphosgene (170 mg, 0.57 mmol) and reacting the resulting dichloromethane concentrated solution of 1-(adamantan-1-yl)ethyl isocyanate with 3-(5-methyl-1H-imidazol-1-yl)propan-1-amine (191 mg, 1.37 mmol), a yellow residue (356 mg) was obtained. Column chromatography purification of this residue (40-60 μm silica gel, dichloromethane-methanol mixtures), followed by washing with pentane (3×1 mL) and drying at 20 Torr for 48 h, afforded the title compound (93 mg, 24% overall yield) as a yellow semi-solid.

Characterization of Ix: IR (ATR) v: 3321, 3109, 2971, 2899, 2846, 2690, 1632, 1556, 1501, 1446, 1378, 1362, 1350, 1319, 1291, 1259, 1233, 1194, 1153, 1108, 1091, 1079, 1052, 1023, 969, 923, 875, 806, 771, 755, 664, 620 cm⁻¹; HRMS (ESI) calcd for [C₂₀H₃₂N₄O+H]⁺ 345.2649, found 345.2654.

### Example 23: Preparation of 1-(adamantan-2-yl)-1-methyl-3-[3-(5-methyl-1H-imidazol-1-yl)propyl]urea, of Formula Iy

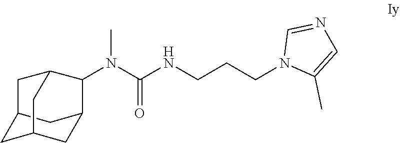 [SMILES: Cc1cncn1CCCNC(=O)N(C)C1C2CC3CC(C2)CC1C3]

To a stirred solution of N-methyladamantan-2-amine (102 mg, 0.62 mmol) and 1-(3-azidopropyl)-5-methyl-1H-imidazole (112 mg, 0.68 mmol) in toluene (10 mL), triphenylphosphine (194 mg, 0.74 mmol) was added. The solution was degassed and a stream of carbon dioxide, obtained from dry ice at room temperature, was bubbled through it, first at room temperature and then at 80° C. for 5.5 h. The resulting mixture was evaporated under reduced pressure to afford a light-orange semi-solid crude (395 mg). Column chromatography purification of this crude (40-60 μm silica gel, dichloromethane-methanol mixtures), followed by washing with pentane (3×2 mL) and drying at 20 Torr for 48 h, afforded the title compound (112 mg, 55% yield) as a white solid.

Characterization of Iy: mp 103-105° C.; IR (ATR) v: 3302, 3109, 3048, 2904, 2850, 2675, 1621, 1526, 1501, 1470, 1449, 1360, 1350, 1334, 1305, 1287, 1266, 1232, 1198, 1169, 1108, 1098, 1060, 1047, 999, 967, 947, 923, 881, 807, 774, 731, 700, 665, 628 cm⁻¹; HRMS (ESI) calcd for [C₁₉H₃₀NaO+H]⁺ 331.2492, found 331.2496.

### Example 24: Preparation of (±)-1-(1-chloroadamantan-2-yl)-3-[3-(5-methyl-1H-imidazol-1-yl)propyl]urea, of Formula Iz

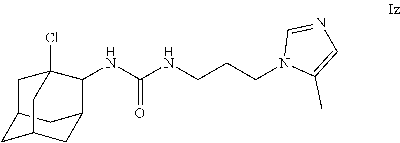 [SMILES: Cc1cncn1CCCNC(=O)NC1C2CC3CC(C2)CC1(Cl)C3]

It was prepared in an analogous manner to the one described for the compound of Example 1. Starting from 1-chloroadamantan-2-amine (133 mg, 0.72 mmol) and triphosgene (106 mg, 0.36 mmol) and reacting the resulting dichloromethane concentrated solution of 1-chloroadamantan-2-yl isocyanate with 3-(5-methyl-1H-imidazol-1-yl)propan-1-amine (120 mg, 0.86 mmol), a dark yellow residue (183 mg) was obtained. Column chromatography purification of this residue (40-60 μm silica gel, dichloromethane-methanol mixtures), followed by washing with pentane (3×1 mL) and drying at 20 Torr for 48 h, afforded the title compound (59 mg, 24% overall yield) as a light yellow sticky oil.

Characterization of Iz: IR (ATR) v: 3301, 3116, 3053, 2912, 2856, 1642, 1557, 1504, 1474, 1451, 1390, 1361, 1338, 1313, 1291, 1257, 1243, 1215, 1161, 1106, 1077, 1066, 1028, 980, 948, 930, 843, 827, 813, 774, 726, 664, 631 cm⁻¹; HRMS (ESI) calcd for [C₁₈H₂₇ClN₄O+H]⁺ 351.1946, found 351.1929.

### Example 25: Preparation of (±)-1-(1-fluoroadamantan-2-yl)-3-[3-(5-methyl-1H-imidazol-1-yl)propyl]urea, of Formula Iaa

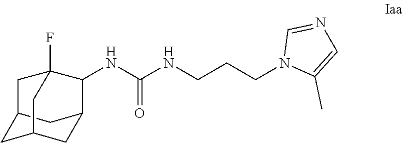 [SMILES: Cc1cncn1CCCNC(=O)NC1C2CC3CC(C2)CC1(F)C3]

It was prepared in an analogous manner to the one described for the compound of Example 1. Starting from 1-fluoroadamantan-2-amine (197 mg, 1.16 mmol) and triphosgene (173 mg, 0.58 mmol) and reacting the resulting dichloromethane concentrated solution of 1-fluoroadamantan-2-yl isocyanate with 3-(5-methyl-1H-imidazol-1-yl)propan-1-amine (194 mg, 1.40 mmol), a yellow residue (332 mg) was obtained. Column chromatography purification of this residue (40-60 μm silica gel, dichloromethane-methanol mixtures), followed by washing with pentane (3×3 mL) and drying at 20 Torr for 48 h, afforded the title compound (120 mg, 31% overall yield) as a yellow sticky oil.

Characterization of Iaa: IR (ATR) v: 3321, 3114, 2914, 2861, 1640, 1556, 1503, 1476, 1451, 1391, 1366, 1343, 1317, 1297, 1261, 1239, 1225, 1148, 1107, 1077, 1060, 982, 960, 931, 912, 883, 829, 814, 781, 732, 699, 664, 630 cm⁻¹; HRMS (ESI) calcd for [C₁₈H₂₇FN₄O+H]⁺ 335.2242, found 335.2235.

### Example 26: Preparation of trans-1-(5-chloroadamantan-2-yl)-3-[3-(5-methyl-1H-imidazol-1-yl)propyl]urea, of Formula Iab

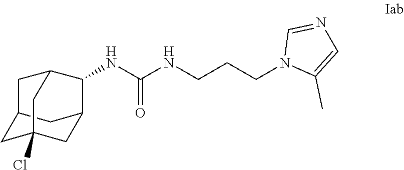 [SMILES: Cc1cncn1CCCNC(=O)N[C@H]1C2CC3CC1C[C@](Cl)(C3)C2]

It was prepared in an analogous manner to the one described for the compound of Example 1. Starting from 5-chloroadamantan-2-amine (189 mg, 1.02 mmol) and triphosgene (151 mg, 0.51 mmol) and reacting the resulting dichloromethane concentrated solution of trans-5-chloroadamantan-2-yl isocyanate with 3-(5-methyl-1H-imidazol-1-yl)propan-1-amine (170 mg, 1.22 mmol), a yellow residue (278 mg) was obtained. Column chromatography purification of this residue (40-60 μm silica gel, dichloromethane-methanol mixtures), followed by washing with pentane (3×3 mL) and drying at 20 Torr for 48 h, afforded the title compound (100 mg, 28% overall yield) as a light yellow solid.

Characterization of Iab: mp 120-122° C.; IR (ATR) v: 3289, 3104, 2915, 2865, 1655, 1567, 1527, 1502, 1475, 1458, 1443, 1428, 1366, 1345, 1331, 1317, 1291, 1268, 1246, 1206, 1197, 1145, 1107, 1092, 1069, 1057, 1039, 1023, 988, 961, 946, 920, 886, 872, 822, 800, 686, 666 cm⁻¹; HRMS (ESI) calcd for [C₁₈H₂₇ClN₄O+H]⁺ 351.1946, found 351.1942.

### Example 27: Preparation of trans-1-(5-fluoroadamantan-2-yl)-3-[3-(5-methyl-1H-imidazol-1-yl)propyl]urea, of Formula Iac

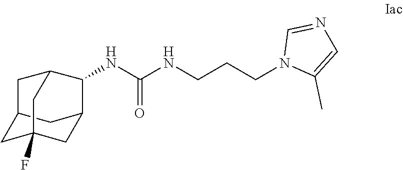 [SMILES: Cc1cncn1CCCNC(=O)N[C@H]1C2CC3CC1C[C@](F)(C3)C2]

It was prepared in an analogous manner to the one described for the compound of Example 1. Starting from 5-fluoroadamantan-2-amine (137 mg, 0.81 mmol) and triphosgene (120 mg, 0.41 mmol) and reacting the resulting dichloromethane concentrated solution of trans-5-fluoroadamantan-2-yl isocyanate with 3-(5-methyl-1H-imidazol-1-yl)propan-1-amine (135 mg, 0.97 mmol), a dark yellow residue (206 mg) was obtained. Column chromatography purification of this residue (40-60 μm silica gel, dichloromethane-methanol mixtures), followed by washing with pentane (3×1 mL) and drying at 20 Torr for 48 h, afforded the title compound (20 mg, 7% overall yield) as a white solid.

Characterization of Iac: mp 198-200° C.; IR (ATR) v: 3322, 3303, 3114, 3053, 3028, 2966, 2916, 2861, 2813, 2716, 2670, 2600, 1830, 1769, 1658, 1618, 1554, 1482, 1460, 1446, 1432, 1400, 1378, 1350, 1334, 1314, 1295, 1267, 1245, 1193, 1184, 1132, 1110, 1074, 1039, 1011, 990, 969, 951, 930, 910, 857, 829, 786, 732, 647, 632, 623 cm⁻¹; HRMS (ESI) calcd for [C₁₈H₂₇FN₄O+H]⁺ 335.2242, found 335.2253.

### Example 28: In Vitro Determination of sEH Inhibitory Activity

The following fluorescent assay was used for determination of the sEH inhibition activity (IC₅₀), with the substrate and comparative control compound (TPPU) indicated below.

Substrate: cyano(6-methoxynaphthalen-2-yl)methyl 2-(3-phenyloxiran-2-yl)acetate (PHOME; from Cayman Chemical, item number 10009134; CAS 1028430-42-3); cf. N. M. Wolf et al., Analitical Biochemistry 2006, vol. 355, pp. 71-80.

Control compound (TPPU): N-[1-(1-Oxopropyl)-4-piperidinyl]-N′-[4-(trifluoromethoxy)phenyl]urea.

### Solutions:

Assay buffer: Bis/Tris HCl 25 mM pH 7.0 containing 0.1 mg/mL of bovine serum albumin (BSA).
PHOME at 200 μM in DMSO.
Solution of recombinant human sEH (hsEH) (Cayman Chemical, item number 10011669), diluted with assay buffer.
Inhibitors dissolved in DMSO at appropriate concentrations.

Protocol: In a black 96-well plate (Greiner Bio-One, item number 655900), the background wells were filled with 90 μL and the positive control and inhibitor wells were filled with 85 μL of assay buffer. Five μL of DMSO were added to background and positive control wells, and then 5 μL of inhibitor solution were added in the inhibitor wells. Five μL of the solution of hsEH were added to the positive control and inhibitor wells and the mixture was stirred. A 1/21 dilution of the solution of PHOME with assay buffer was prepared according to the final volume required, and then 105 μL of each well were added. The plate was carefully shaken for 10 seconds and was incubated for 5 minutes at room temperature. The appearance of fluorescence was read with excitation wavelength: 337 nm, and emission wavelength: 460 nm (FLUOStar OPTIMA microplate reader, BMG). The intensity of fluorescence was used to analyze and calculate the IC₅₀ values. Results were obtained by regression analysis from at least three data points in a linear region of the curve. IC₅₀ values are average of minimum three independent replicates. Table 1 shows the human hsEH inhibitory activities (expressed as IC₅₀) of several compounds of formula I and of control compound TPPU.

TABLE 1
Human sEH inhibitory activity of compounds of formula I

| | IC₅₀ | | IC₅₀ |
|---|---|---|---|
| Compound | (μM) | Compound | (μM) |
| TPPU | 0.0037 | Ik | 0.0090 |
| Ia | 0.035 | Im | 0.30 |
| Ib | 0.093 | Iw | 0.072 |
| Ic | 0.0133 | Io | 0.0013 |
| Id | 0.0033 | Ip | 0.095 |
| Ie | 0.029 | Iq | 5.1 |
| If | 0.037 | Ir | 0.86 |
| Ig | 0.150 | Is | 2.8 |
| Ih | 0.33 | It | 0.185 |
| Ii | 0.0043 | Iv | 0.103 |
| Ij | 0.045 | Iu | 0.194 |
| Ix | 0.059 | Iab | 0.039 |
| Iz | 0.030 | Iac | 0.020 |
| Iaa | 5.9 | Iy | 0.023 |

### Example 29: In Vitro Determination of Human QC Inhibitory Activity

The compounds were assessed for their ability to inhibit human recombinant glutaminyl cyclase (hQC) using SensoLyte® Green Glutaminyl Cyclase Activity Assay Kit Fluorimetric (Eurogentec, AS-72230), according to the manual. The compounds were tested at 7 concentrations, with each condition being assessed in triplicates.

The inhibitors to be tested were dissolved in DMSO, as a 250 mM stock solution and stored at −20° C. until further use. For the assay, compounds were diluted in the provided assay buffer to the desired concentrations, from 100 μM to 0.1 nM, so that in all wells a final DMSO concentration of 0.6% was present. The enzyme solution was prepared immediately before use, by diluting 10 μL of recombinant enzyme (component C) in 3.99 mL of assay buffer (component E) and it was stored on ice. The substrate solution was prepared by diluting 50 μL of glutaminyl cyclase substrate (0.5 mM, component A) in 4.95 mL of assay buffer (component E). The developer solution was prepared by diluting 50 μL of glutaminyl cyclase developer (component D) and 50 μL of 1-benzylimidazole (component F) in 4.9 mL of assay buffer (component E), and was kept on ice until use.

In brief, 10 μL of the solution of the inhibitor were placed into wells of a black 96-well plate and combined with 40 μL of the diluted enzyme solution. To start the reaction 50 μL of glutaminyl cyclase substrate solution were added. After 30 min incubation at 37° C., 50 μL of developer solution were added and mixed. After another 30 min incubation at 37° C., the signal was measured at EX485/EM535 at Cytation 5 multimode reader. Vehicle control (0.6% DMSO without inhibitor), substrate control (no enzyme solution, only assay buffer) and inhibitor control (1 mM 1-benzylimidazole) were present on each plate. The % activity of vehicle control were calculated and used for assessment of IC₅₀ in GraphPad Prism 9 (log(inhibitor) vs. response—Variable slope (four parameters)).

Basic statistical analysis was performed. Table 2 shows the hQC inhibitory activity of several compounds of formula I and of control compound 1-[3-(1H-imidazol-1-yl)propyl]-3-(3,4-dimethoxyphenyl)thiourea (PBD-150).

TABLE 2
Human QC inhibitory activity of compounds of formula I

| | IC₅₀ | | IC₅₀ |
|---|---|---|---|
| Compound | (μM) | Compound | (μM) |
| PBD-150 | 0.050 | Ik | 0.73 |
| Ia | 0.61 | Im | 3.5 |
| Ib | 12.7 | Iw | 6.8 |
| Ic | 1.24 | Io | 1.65 |
| Id | 0.57 | Ip | 1.45 |
| Ie | 2.1 | Iq | 0.190 |
| If | 0.99 | Ir | 0.139 |
| Ig | 0.81 | Is | 0.096 |
| Ih | 1.04 | It | 0.46 |
| Ii | 0.189 | Iv | 0.54 |
| Ij | 5.2 | Iab | 0.559 |
| Iz | 0.496 | Iac | 0.236 |
| Iaa | 0.767 | Iy | 0.205 |

### Example 30: Effects of Compounds of Formula I on Cytokine Release in LPS Stimulated BV2 Cells

Compounds of formula I were diluted from 250 mM DMSO stock solutions to a 10 mM stock in DMSO. Thereafter, the compounds were diluted to 10 μM and 1 μM in medium, so that the final DMSO concentration in the well was 0.1% for all conditions. The stock solutions were stored at −20° C. until further use.

Culture and treatment of BV-2 cells: The murine microglial cell line BV-2 was cultivated in Dulbecco's modified eagle medium (DMEM) supplemented with 10% fetal calf serum (FCS), 1% penicillin/streptomycin and 2 mM L-glutamine (culture medium). For the lipopolysaccharide (LPS) stimulation assay, 20,000 BV-2 cells per well (uncoated 96 well plates) were plated out. After 48 h, the medium was changed to treatment medium (DMEM, 5% FCS, 2 mM L-glutamine) and cells were maintained in treatment medium for the remaining culture period. After changing cells to treatment medium, the compounds were administered 1 h before LPS stimulation (Sigma-Aldrich; L6529; 1 mg/mL stock in ddH₂O, final concentration in well: 100 ng/mL (dilutions in medium)). Cells treated with vehicle and LPS alone served as controls. All wells were handled the same way. After 24 h of stimulation, cell supernatants were collected for the cytokine measurement. The experiment was performed in n=6 technical replicates for all groups.

Cytokine measurement: The samples were diluted 1:2 and analysed for 4 cytokines (IL-1β, IL-2, IL-6, TNF-α) with a commercially available immunosorbent assay kit (U-plex, K15069L, Mesoscale Discovery) according to the instructions of the manufacturer. In brief, 25 μL of diluent 41 and 25 μL sample or respective standard were placed into wells of the pre-coated U-plex plate and incubated on an orbital shaker at room temperature for 1 h. Plates were washed 3 times with phosphate buffered saline with 0.05% Tween 20 (PBST) and thereafter 50 μL of detection antibody were added. Plates were again incubated on an orbital shaker at room temperature for 1 h. After another 3 washing steps with PBST, 150 μL of Mesoscale Discovery (MSD) GOLD Read Buffer were added and plates were read on Sector imager. Data were evaluated in comparison to calibration curves provided in the kit.

Statistics: Basic statistical analysis was performed. Group differences were evaluated by e.g. one-way ANOVA followed by a posthoc test, if appropriate.

Table 3 shows the effect of compounds of formula I, at 1 μM and 10 μM concentration, on the levels of proinflammatory cytokines (TNF-α, IL-6, IL-2 and IL-1β) in LPS stimulated BV2 cells, expressed as % of vehicle control, using TPPU and dexamethasone as controls.

TABLE 3
Effect of compounds of formula I on the
levels of proinflammatory cytokines

| | Concentration | | | | |
|---|---|---|---|---|---|
| Compound | (μM) | TNF-α | IL-6 | IL-2 | IL-1β |
| Ii | 1 | 104 | 63 | 66 | 86 |
| | 10 | 103 | 62 | 48 | 125 |
| Is | 1 | 104 | 65 | 66 | 95 |
| | 10 | 104 | 64 | 48 | 124 |
| Id | 1 | 98 | 34 | 38 | 46 |
| | 10 | 84 | 24 | 26 | 73 |
| Ir | 1 | 107 | 37 | 52 | 77 |
| | 10 | 102 | 38 | 37 | 94 |
| dexamethasone | 10 | 49 | 55 | 50 | 42 |
| TPPU | 10 | 96 | 117 | 113 | 125 |

### Example 31: Effects of Compounds of Formula I on the Viability of Mouse Primary Neurons

The compounds were diluted from 250 mM DMSO stocks to a 100 mM stock in DMSO. Thereafter, compounds were diluted to 100 μM, 20 μM, 5 μM, 1 μM, 0.2 μM, 0.05 μM, 0.01 μM and 0.001 μM dilution in medium, so that the final DMSO concentration in the well was 0.1% for all conditions. Stock solutions were stored at −20° C. until further use.

Isolation and treatment of primary mouse cortical neurons: Primary cortical neurons were prepared from timed pregnant wild-type C57BL/6JRccHsd mice at E16.5-E18. Animals were sacrificed by carbon dioxide and embryos were dissected in Calcium and Magnesium free Hanks Balanced Salt Solution (CMF-HBSS) containing 15 mM HEPES and 10 mM NaHCO₃, pH 7.2. Embryos were decapitated, skin and skull gently removed and cortical hemispheres were separated. After removing meninges and brain stem, the cortex was isolated, chopped with a sterile razor blade in Chop solution (Hibernate-E without Calcium containing 2% B-27) and digested in 2 mg/mL papain (Worthington) dissolved in Hibernate-E without Calcium for 30 min at 30° C. Cortices were triturated for 10-15 times with a fire-polished silanized Pasteur pipette in Hibernate-E without Calcium containing 2% B-27, 0.01% DNaseI, 1 mg/mL BSA, and 1 mg/mL Ovomucoid Inhibitor. Undispersed pieces were allowed to settle by gravity for 1 min and the supernatant was centrifuged for 3 min at 228 g. The pellet was resuspended in Hibernate-E containing 2% B-27, 0.01% DNaseI, 1 mg/mL BSA, 1 mg/mL Ovomucoid Inhibitor and diluted with Hibernate-E containing 2% B-27. After the second centrifugation step (3 min at 228 g), the pellet was resuspended in nutrition medium without glutamate (Neurobasal, 2% B-27, 0.5 mM glutamine, 1% Penicillin-Streptomycin). Cells were counted in a hemacytometer and seeded in nutrition medium on poly-D-lysine pre-coated plates. Cells were cultured at 37° C.; 95% humidity and 5% CO₂. All wells were handled the same way. On DIV8, cells were treated with the compounds for the evaluation of the neurotoxic effect. On DIV9, the MTT and LDH assays were performed as described below. The experiments were performed in six technical replicates for all groups per experiment.

MTT viability assay: Viability of cultures was determined on DIV9 by the MTT assay using a plate-reader (570 nm). MTT solution was added to each well in a final concentration of 0.5 mg/mL. After 2 h the MTT containing medium was aspirated. Cells were lysed in 3% SDS and the formazan crystals were dissolved in isopropanol/HCl. Optical density was measured with a plate-reader at wavelength 570 nm. Cell survival rate was expressed as optical density (OD). Values were calculated as percent of control values (vehicle control).

LDH toxicity assay: Supernatants of cells were subjected to the lactate dehydrogenase (LDH) toxicity assay by using the Cytotoxicity Detection Kit (Roche Diagnostics, Cat. No: 11 644 793 001). Seventy μL of cell culture supernatant was transferred to clear 96-well plates. Seventy μL freshly prepared reaction mixture was added to each well and the mixture was incubated for 20 to 30 min at room temperature protected from light. Absorbance was measured at 492 nm and 620 nm as reference wavelength. Cell death was expressed as optical density (OD). Values were calculated as percent of control values (vehicle control).

Table 4 shows the primary cortical neuronal cell viability after treatment with different concentrations of compounds of formula I, assessed by both the MTT and the LDH assays, expressed as % of vehicle control.

TABLE 4
Neurotoxicity assessment of compounds of formula I: primary cortical
neuronal cell viability expressed as % of vehicle control

| | | Compound concentration (μM) | | | | | | | |
|---|---|---|---|---|---|---|---|---|---|
| Compd. | | 0.001 | 0.01 | 0.05 | 0.2 | 1 | 5 | 20 | 100 |
| Ii | MTT | 101 | 95 | 99 | 96 | 101 | 101 | 101 | 55 |
| | LDH | 99 | 97 | 97 | 94 | 101 | 100 | 97 | 113 |
| Is | MTT | 104 | 102 | 103 | 96 | 101 | 103 | 101 | 86 |
| | LDH | 96 | 97 | 99 | 98 | 107 | 102 | 113 | 107 |
| Id | MTT | 110 | 107 | 100 | 99 | 99 | 93 | 89 | 53 |
| | LDH | 114 | 104 | 100 | 100 | 99 | 102 | 109 | 157 |
| Ir | MTT | 97 | 97 | 93 | 99 | 101 | 99 | 99 | 87 |
| | LDH | 100 | 100 | 101 | 101 | 100 | 100 | 101 | 107 |

### Example 32: Parallel Artificial Membrane Permeation Assay—Blood-Brain Barrier

A parallel artificial membrane permeation assay for blood-brain barrier (PAMPA-BBB) was used to evaluate the brain penetration of compounds of formula I, following the method described by L. Di et al., “High throughput artificial membrane permeability assay for blood-brain barrier”, European Journal of Medicinal Chemistry 2003, vol. 38. pp. 223-232. The in vitro permeability (Pe) of the test compounds through a lipid extract of porcine brain membrane was determined. Assayed compounds were tested using a mixture of PBS:EtOH (70:30). Assay validation was made by comparing the experimental and reported permeability values of a set of fourteen commercial drugs and a lineal correlation between experimental and reported permeability values of the fourteen commercial drugs was obtained (y=1.678 x−1.447; R²=0.9313). From this equation and taking into account the limits established by Di et al. for BBB permeation, the ranges of permeability were established, as follows. Compounds of high BBB permeation (CNS+): Pe (10⁻⁶ cm s⁻¹)>5.26; compounds of low BBB permeation (CNS−): Pe (10⁻⁶ cm s⁻¹)<1.91; and compounds of uncertain BBB permeation (CNS+/−): 5.26>Pe (10⁻⁶ cm s⁻¹)>1.91. The permeability results are averages of three different experiments in triplicate and a predictive penetration in the CNS is also given (Table 5).

TABLE 5
Blood-brain barrier permeability values (Pe in
10⁻⁶ cm s⁻¹) from the PAMPA-BBB assay

| | | CNS | | | CNS |
|---|---|---|---|---|---|
| Compound | Pe | prediction | Compound | Pe | prediction |
| Ia | 7.1 | CNS+ | Ik | 14.1 | CNS+ |
| Iu | 5.4 | CNS+ | Im | 11.8 | CNS+ |
| Ic | 10.2 | CNS+ | Ix | 6.5 | CNS+ |
| Id | >30 | CNS+ | Io | 18.7 | CNS+ |
| Ie | 8.8 | CNS+ | Ip | 13.0 | CNS+ |
| Ij | 9.4 | CNS+ | Iq | 26.4 | CNS+ |
| It | 5.8 | CNS+ | Ir | 6.9 | CNS+ |
| Iv | 7.5 | CNS+ | Is | >30 | CNS+ |
| Ii | >30 | CNS+ | Iy | 9.8 | CNS+ |
| Iz | 6.0 | CNS+ | Iab | 13.1 | CNS+ |
| Iaa | 7.4 | CNS+ | Iac | 8.6 | CNS+ |

1. A compound of formula I, a stereoisomer or a mixture of stereoisomers thereof, or a pharmaceutically acceptable salt of any of them, 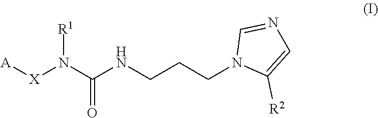
wherein:
R¹ and R² are each one a radical independently selected from the group consisting of H and (C1-C3)-alkyl;
X is a biradical selected from the group consisting of a biradical of a linear methylene chain of formula —[CH₂]ₙ— with n=0, 1 or 2, and a biradical from a branched saturated (C2-C4)-alkylene chain; and
A is either a C-radical from a non-aromatic polycyclic 6- to 15-membered carbocyclic ring system, or a C-radical from a non-aromatic polycyclic 6- to 15-membered heterocyclic ring system having one or two heteroatoms in the ring, the heteroatoms being independently selected from O, S and N; wherein the C-radicals are unsubstituted or the C-radicals have one or more of their H atoms substituted by one or more substituents that are attached to any of the possible substitution positions, the substituents being independently selected from the group consisting of (C1-C4)-alkyl, O[(C1-C4)-alkyl], OH, F, Cl, Br, and I;
on the proviso that A does not have any one of the three accompanying unsubstituted formulas, nor any of the possible substituted formulas derived thereof, and 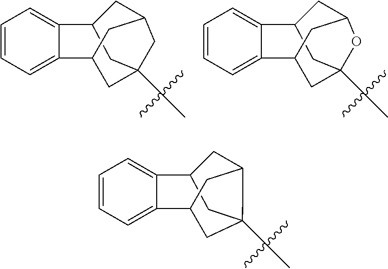 [SMILES: *C12CC3CC(C1)c1ccccc1C(C3)C2 ; *C12CC3CC(CC(C1)c1ccccc13)O2 ; *C12CC3CC1CC(C2)c1ccccc13]
on the proviso that the compound of formula I does not have the accompanying formula Ib

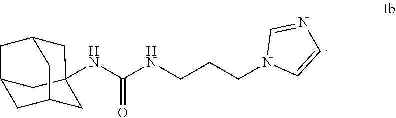 [SMILES: O=C(NCCCn1ccnc1)NC12CC3CC(CC(C3)C1)C2]

2. The compound of claim 1, wherein A has a formula which is one of the unsubstituted accompanying formulas, or A has a formula which is a modification of the accompanying formulas in which one or more of the H atoms are substituted by one or more substituents which are attached to any of the possible substitution positions, and which are independently selected from the group consisting of (C1-C4)-alkyl, O[(C1-C4)-alkyl], OH, F, Cl, Br, and I

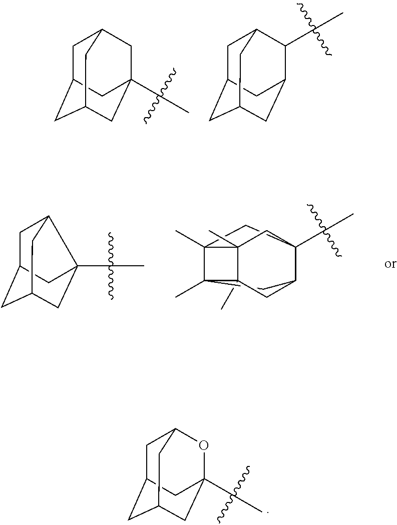

3. The compound of claim 2, wherein A has one of the unsubstituted indicated formulas, or A has a formula which is a modification of the accompanying formulas in which one or more of the H atoms are substituted by one or more substituents which are attached to any of the possible substitution positions, and which are independently selected from the group consisting of CH₃, CH₂CH₃, OH, F, and Cl.

4. The compound of claim 1, wherein biradical X is selected from the group consisting of: a biradical of a linear methylene chain of formula —[CH₂]ₙ— with n=0, 1 or 2; and the biradical —CH(CH₃)—.

5. The compound of claim 4, wherein biradical X is a biradical of a linear methylene chain of formula —[CH₂]ₙ— with n=0, 1 or 2.

6. The compound of claim 5, wherein n=0, biradical X being a single bond.

7. The compound of claim 1, wherein R¹ is H or CH₃.

8. The compound of claim 7, wherein R¹ is H.

9. The compound of claim 1, wherein R² is H or CH₃.

10. The compound of claim 9, wherein R² is CH₃.

11. The compound of claim 1, having a formula independently selected from the group consisting of accompanying formulas Ia, Ic, Id, Ie, If, Ig, Ih, Ii, Ij, Ik, Im, Io, Ip, Iq, Ir, Is, It, Iu, Iv, Iw, Ix, Iy, Iz, Iaa, Iab and Iac

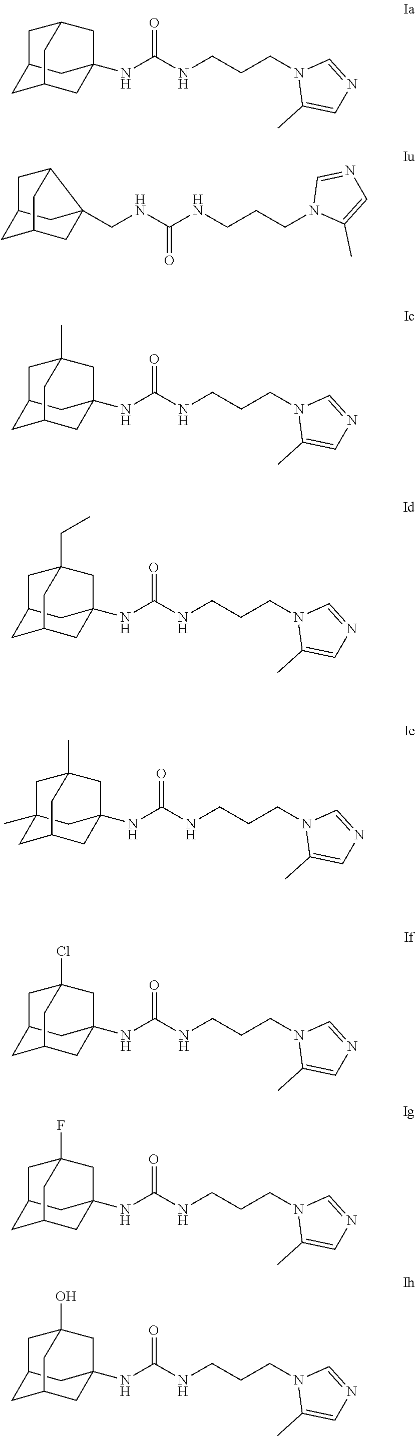

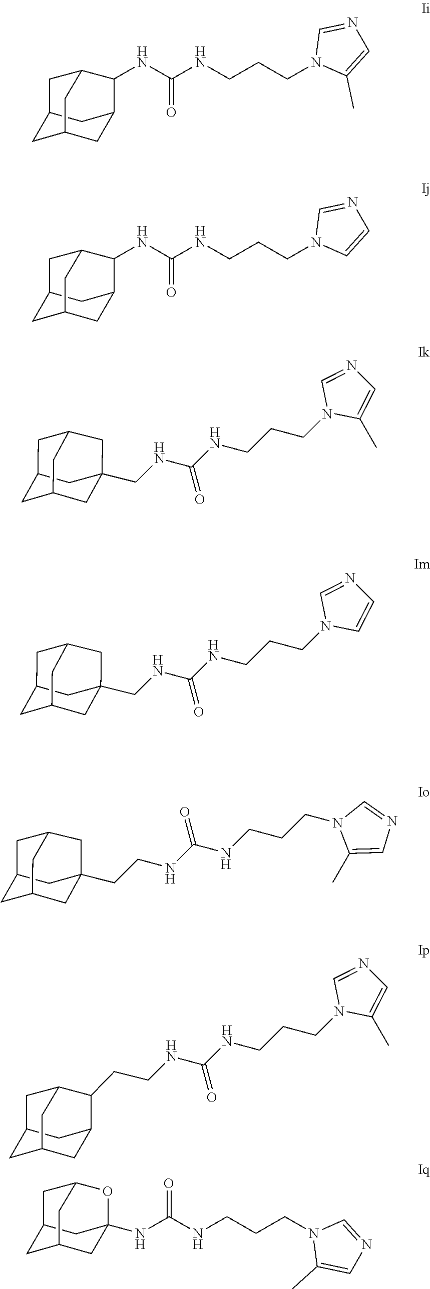

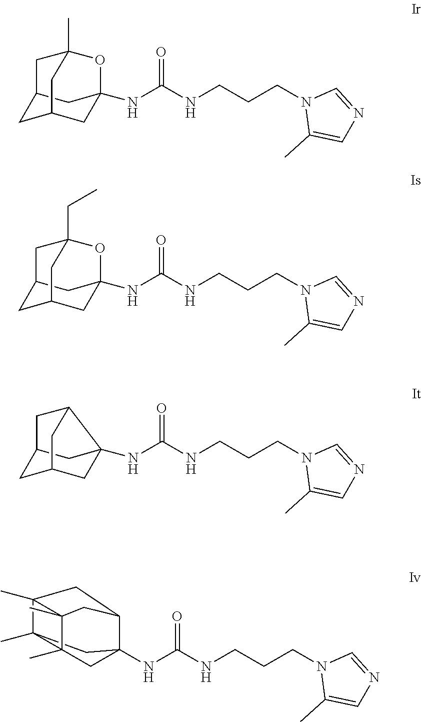 [SMILES: Cc1cncn1CCCNC(=O)NC12CC3CC(CC(C)(C3)O1)C2 ; CCC12CC3CC(C1)CC(NC(=O)NCCCn1cncc1C)(C3)O2 ; Cc1cncn1CCCNC(=O)NC12CC3CC(CC1C3)C2 ; Cc1cncn1CCCNC(=O)NC12CC3(C)C4(C)CC1CC4(C)C3(C)C2]

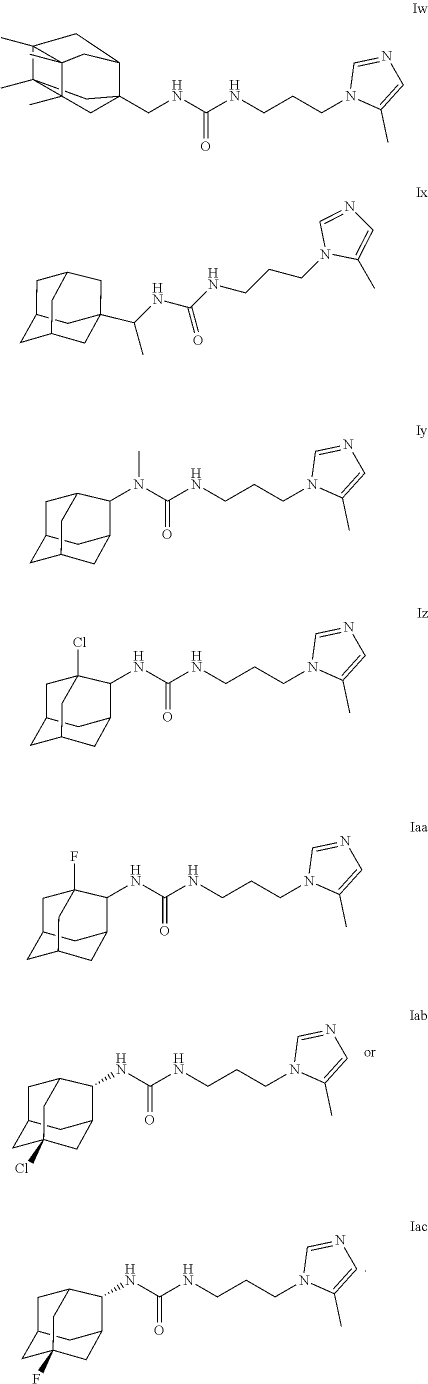

12. A pharmaceutical composition comprising an effective amount of the compound of claim 1, together with adequate amounts of pharmaceutically acceptable excipients or carriers.

13. (canceled)

14. A method of treating or preventing Alzheimer's disease in an animal, the method comprising administering the compound of claim 1 or the compound of formula Ib:

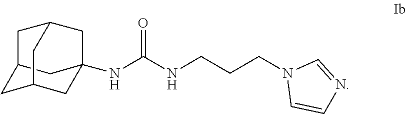 [SMILES: O=C(NCCCn1ccnc1)NC12CC3CC(CC(C3)C1)C2]

15. The compound of claim 2, wherein biradical X is selected from the group consisting of: a biradical of a linear methylene chain of formula —[CH₂]ₙ— with n=0, 1 or 2; and the biradical —CH(CH₃)—.

16. The compound of claim 3, wherein biradical X is selected from the group consisting of: a biradical of a linear methylene chain of formula —[CH₂]ₙ— with n=0, 1 or 2; and the biradical —CH(CH₃)—.

17. The compound of claim 2, wherein R¹ is H or CH₃.

18. The compound of claim 3, wherein R¹ is H or CH₃.

19. The compound of claim 4, wherein R¹ is H or CH₃.

20. The compound of claim 5, wherein R¹ is H or CH₃.

21. The compound of claim 6, wherein R¹ is H or CH₃.
